(19) United States
(12) Patent Application Publication
(10) Pub. No.: US 2025/0001101 A1
(43) Pub. Date: Jan. 2, 2025

(54) OPTICAL SENSING MODULE AND NEBULIZER INCLUDING THE SAME

(71) Applicant: HCMed Innovations Co., LTD., Taipei City (TW)

(72) Inventors: CHUN-YING LI, New Taipei City (TW); SHIH-CHAO LUO, New Taipei City (TW); HSIN-YI PAI, New Taipei City (TW); CHIEN-SHEN TSAI, New Taipei City (TW)

(21) Appl. No.: 18/759,242

(22) Filed: Jun. 28, 2024

Related U.S. Application Data
(60) Provisional application No. 63/524,199, filed on Jun. 29, 2023.

(30) Foreign Application Priority Data
Jun. 19, 2024 (CN) 202410788720.2

(51) Int. Cl. A61M 11/00

(52) U.S. Cl. CPC A61M 11/00; A61M 2016/0021; A61M 2205/3306

(57) ABSTRACT

A nebulizer includes a host, a cup body, a nozzle assembly, a nebulizing module, and an optical sensing module. The host has a detection hole. The cup body is disposed on a top of the host. The nozzle assembly is disposed on the cup body. The nozzle assembly has a nozzle. The nebulizing module is disposed on the cup body. The optical sensing module includes an optical sensor and a membrane structure. The optical sensor is disposed inside the host. The membrane structure is disposed in the detection hole, and the membrane structure blocks between the nozzle and the optical sensor. When a user inhales against the nozzle, the membrane structure is deformed by a pressure difference generated inside the nozzle, and the optical sensor is used to detect a deformation amount of the membrane structure.

### CROSS-REFERENCE TO RELATED PATENT APPLICATION

This application claims the benefit of priority to the U.S. Provisional Patent Application Ser. No. 63/524,199 filed on Jun. 29, 2023, which application is incorporated herein by reference in its entirety.

This application claims the benefit of priority to China Patent Application No. 202410788720.2, filed on Jun. 19, 2024 in People's Republic of China. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to an optical sensing module and a nebulizer having the same, and more particularly to an optical sensing module and a nebulizer having the same that can prevent moisture from affecting breathing detection.

### BACKGROUND OF THE DISCLOSURE

Nebulizers for medical use mainly deliver drugs through the respiratory system. The nebulizers atomize the medicinal liquid into small particles with a specific particle size and deliver the small particles from the patient's mouth and nose into the respiratory system of the patient's body through breathing for treatment purposes.

The existing nebulizer uses an internal pressure sensor configured therein for breathing detection. The pressure sensor is in intercommunication with an external environment through a detection hole on the nebulizer host. When a user holds a nozzle of the nebulizer in his mouth and inhales, the pressure sensor can detect, through the detection hole, the change in air pressure (i.e., pressure difference generated in the nozzle) during the breathing. However, during this process, the aerosol generated by the medicinal liquid or the liquid generated inside the user's mouth and nose may penetrate into the nebulizer host through the detection hole, causing a short circuit or malfunction of the circuit board in the nebulizer host. In addition, in the existing technology, the pressure sensor needs to read an initial pressure value before using the nebulizer for breathing detection. However, the reading of the initial pressure value varies depending on whether the user's mouth completely covers the nozzle, and the difference in the initial pressure value may cause the breathing detection function to operate incorrectly.

On the other hand, in the related art, when the user holds the nebulizer for operation, an external force is exerted on the nebulizer due to the user's handgrip, which generates an internal pressure in the nebulizer host and easily produces an error during measurement, thereby affecting the accuracy of the measured result.

### SUMMARY OF THE DISCLOSURE

In response to the above-referenced technical inadequacies, the present disclosure provides an optical sensing module and a nebulizer comprising the same, which can not only prevent moisture from penetrating into the nebulizer and causing damage inside an electronic device, but also prevent the accuracy of the measurement results from being affected by pressure generated inside the nebulizer host.

In order to solve the above-mentioned problems, one of the technical aspects adopted by the present disclosure is to provide an optical sensing module applied for a nebulizer. The nebulizer has a nozzle. The optical sensing module includes an optical sensor and a membrane structure. The optical sensor is disposed inside the nebulizer. The membrane structure is disposed in the nebulizer, and the membrane structure blocks between the nozzle and the optical sensor. When a user inhales against the nozzle, the membrane structure is deformed by a pressure difference generated inside the nozzle, and the optical sensor is used to detect a deformation amount of the membrane structure.

In order to solve the above-mentioned problems, another one of the technical aspects adopted by the present disclosure is to provide a nebulizer includes a host, a cup body, a nozzle assembly, a nebulizing module, and an optical sensing module. The host has a detection hole. The cup body is disposed on a top of the host. The nozzle assembly is disposed on the cup body. The nozzle assembly has a nozzle. The nebulizing module is disposed on the cup body. The optical sensing module includes an optical sensor and a membrane structure. The optical sensor is disposed inside the host. The membrane structure is disposed in the detection hole, and the membrane structure blocks between the nozzle and the optical sensor. When a user inhales against the nozzle, the membrane structure is deformed by a pressure difference generated inside the nozzle, and the optical sensor is used to detect a deformation amount of the membrane structure.

Therefore, in the optical sensing module and the nebulizer provided by the present disclosure, by virtue of the membrane structure being disposed in the detection hole, the optical sensor can be used to detect a deformation amount of the membrane structure, the optical sensor is used to detect a deformation amount of the membrane structure to control whether the nebulizing module is activated. In the nebulizer of the present disclosure, the membrane structure blocks between the nozzle and the optical sensor, preventing moisture in the external environment from penetrating into the inside of the nebulizer through the detection hole. Moreover, in the nebulizer of the present disclosure, the optical sensor replaces the existing pressure sensor for measuring pressure changes. The optical sensor is used to detect the deformation of the membrane structure to determine the user's breathing status and activates a nebulizing module when the user inhales.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1 is a schematic view of a first embodiment of a nebulizing module according to the present disclosure;

FIG. 2 is a schematic view of a second embodiment of the nebulizing module according to the present disclosure;

FIG. 3 is a schematic exploded view of the first embodiment of a nebulizer according to the present disclosure;

FIG. 4 is a schematic internal view of the first embodiment of the nebulizer according to the present disclosure; and

FIG. 5 is a schematic exploded view of the second embodiment of the nebulizer according to the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a,” “an” and “the” includes plural reference, and the meaning of “in” includes “in” and “on.” Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first,” “second” or “third” can be used to describe various components, signals or the like, which are for distinguishing one component/signal from another one only, and are not intended to, nor should be construed to impose any substantive limitations on the components, signals or the like.

### Embodiments

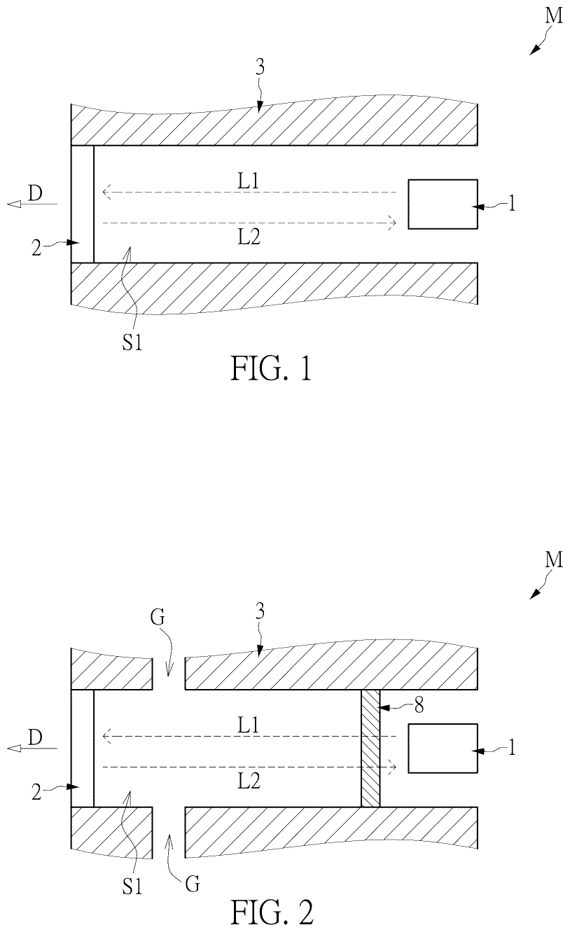

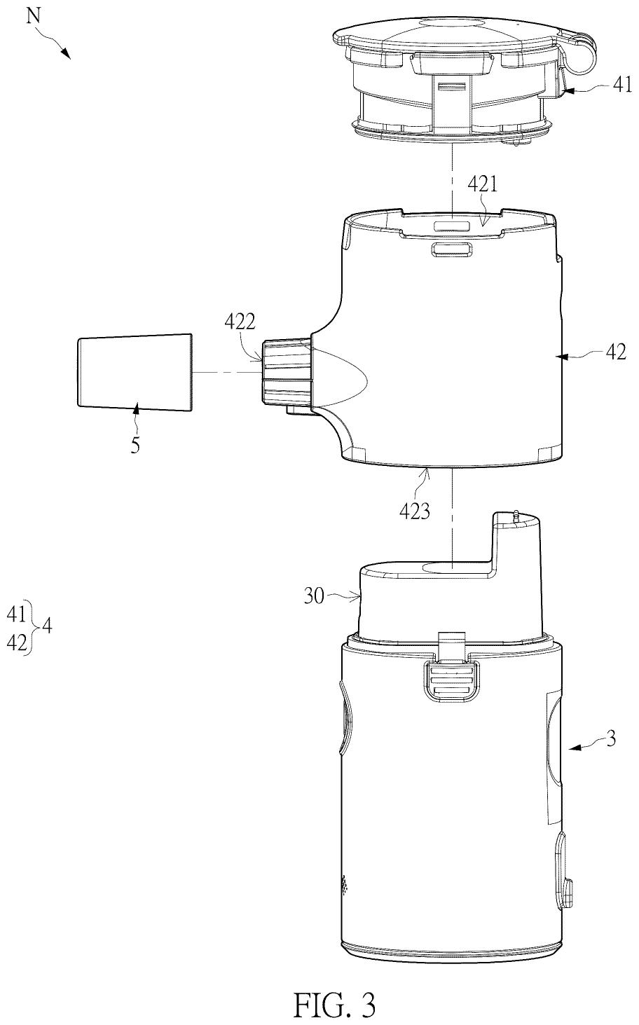

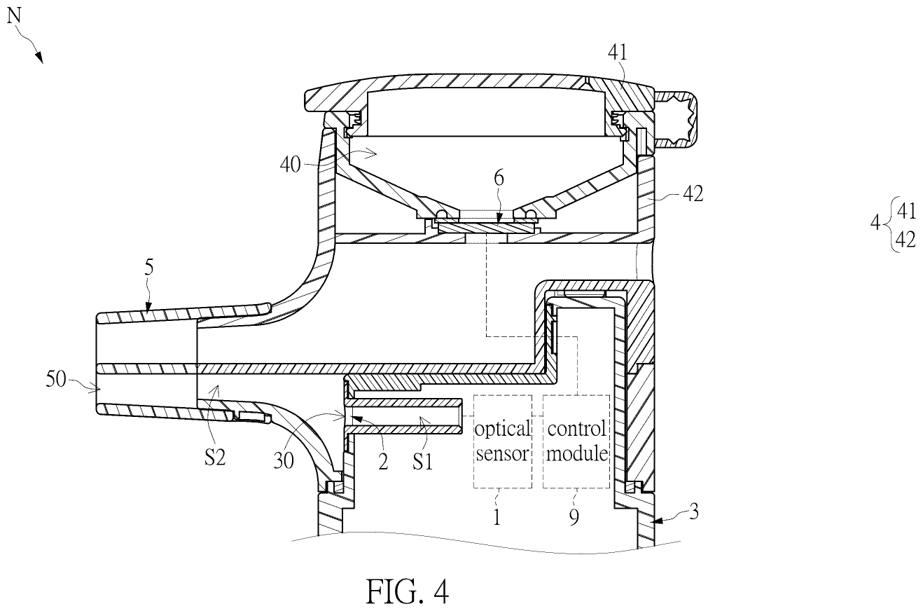

Reference is made to FIG. 1, FIG. 3, and FIG. 4. The present disclosure provides a nebulizer N including an optical sensing module M. The optical sensing module M is disposed inside the nebulizer N. The optical sensing module M includes an optical sensor 1 and a membrane structure 2. The relative position between the optical sensor 1 and the membrane structure 2 is shown in FIG. 1. The nebulizer N further includes a host 3, a cup body 4, a nozzle assembly 5, and a nebulizing module 6. The cup body 4 is disposed on a top of the host 3. The nozzle assembly 5 and the nebulizing module 6 are disposed on the cup body 4. The host 3 has a detection hole 30. The nozzle assembly 5 has a nozzle 50.

Reference is further made to FIG. 3 and FIG. 4. The cup body 4 includes a first housing 41 and a second housing 42. The first housing 41 is capable of storing medical liquid. The second housing 42 includes a first connection opening 421, a second connection opening 422, and a third connection opening 423 that are in fluid communication with each other. The first housing 41 is disposed on the first connection opening 421. The nozzle assembly 5 is disposed on the second connection opening 422. The second housing 42 is coupled to a head of the host 3 through the third connection opening 423. The nebulizing module 6 is disposed on a bottom of the first housing 41. The optical sensor 1 is disposed inside the host 3 of the nebulizer N. The membrane structure 2 is disposed in the detection hole 30 of the host 3 and is used to seal the detection hole 30.

The optical sensor 1 is a sensor that uses a photosensitive element to convert optical signals into electrical signals. The specific implementation of the optical sensor 1 is not limited in the present disclosure. For example, the optical sensor 1 can be composed of a light-emitting portion that transmits light and a light-receiving portion that receives the light, which is not shown in the figures. When the light emitted by the light-emitting portion is reflected by an object to be detected and received by the light-receiving portion, the amount of light that is received can vary. Accordingly, the light-receiving portion outputs a sensing signal after detecting the change in the amount of light received.

Reference is made to FIG. 4. When the cup body 4, the nozzle assembly 5, and the host 3 are assembled together, the membrane structure 2 and the optical sensor 1 have a detection channel S1 therebetween, and the membrane structure 2 and the nozzle 50 have a gas diffusion channel S2 therebetween. The gas diffusion channel S2 and the detection channel S1 are separated from and not in fluid communication with each other. Since the membrane structure 2 is disposed in the detection hole 30 of the host 3, an internal space of the host 3 cannot be in fluid communication with the external environment through the detection hole 30. Therefore, through arranging the membrane structure 2 in the detection hole 30 of the host 3, the membrane structure 2 blocks between the nozzle 50 and the optical sensor 1, and the optical sensor 1 is isolated from the external environment, so as to prevent water droplets or moisture from the external environment from penetrating into the host 3 and causing a short circuit or malfunction of electronic components in the host 3.

Reference is made to FIGS. 1 and 4. When the user holds the nozzle 50 of the nozzle assembly 5 in the mouth and inhales (a direction D in FIG. 1 indicates the direction toward the nozzle 50), a pressure difference is generated between the gas diffusion channel S2 and the detection channel S1 due to the user's breathing, and the pressure difference causes the membrane structure 2 to deform. Meanwhile, the optical sensor 1 outputs light L1 through the detection channel S1 and receives reflected light L2 to detect the deformation of the membrane structure 2 and output the aforementioned sensing signal accordingly. For example, the material of the membrane structure 2 includes an elastomer, and a thickness of the membrane structure 2 ranges between 0.01 and 0.05 mm. In another embodiment, the membrane structure 2 can be a cross-sectional structure in which a central region is thinner than a peripheral region. In addition, the nebulizer N further includes a control module 9, which is electrically connected to the nebulizing module 6 and the optical sensor 1. The control module 9 can receive the sensing signal and send a control signal accordingly, thereby controlling the activation of the nebulizing module 6 to perform a nebulizing action.

For example, when the nebulizer N is not activated, the user can hold the nozzle assembly 5 in the mouth tightly and inhale against the nozzle 50, so that the gas in the gas diffusion channel S2 flows toward the user's mouth, thereby generating an outward negative pressure. The negative pressure causes the membrane structure 2 to produce a deformation amount, and the optical sensor 1 is used to sense the deformation amount to generate a sensing signal and transmit to the control module 9. In response to the sensing signal, the control module 9 outputs an activation signal and transmit to the nebulizing module 6. After receiving the activation signal, the nebulizing module 6 performs a nebulizing action to nebulize the medicinal liquid in the first housing 41 of the cup body 4 to a space below the nebulizing module 6, such that the medicinal liquid in aerosol form is inhaled by the user through the nozzle assembly 5.

On the other hand, when the user stops inhaling or moves his mouth away from the nozzle assembly 5, the gas inside the gas diffusion channel S2 does not flow, such that the optical sensor 1 does not sense any pressure change. Without the sensing signal being provided to the control module 9, the control module 9 does not output any signal to the nebulizing module 6, and the nebulizing module 6 does not perform the nebulizing action.

Reference is made to FIG. 2. FIG. 2 shows another implementation of the nebulizing module 6. The detection channel S1 is not necessarily in a sealed state. The detection channel S1 has a gap G to balance the pressure on both sides of the membrane structure 2. However, the moisture can penetrate through the gap G simultaneously. Therefore, the detection channel S1 can be provided with a transparent barrier layer 8 to block the moisture from entering the detection channel S1 without affecting the direction of the lights L1 and L2.

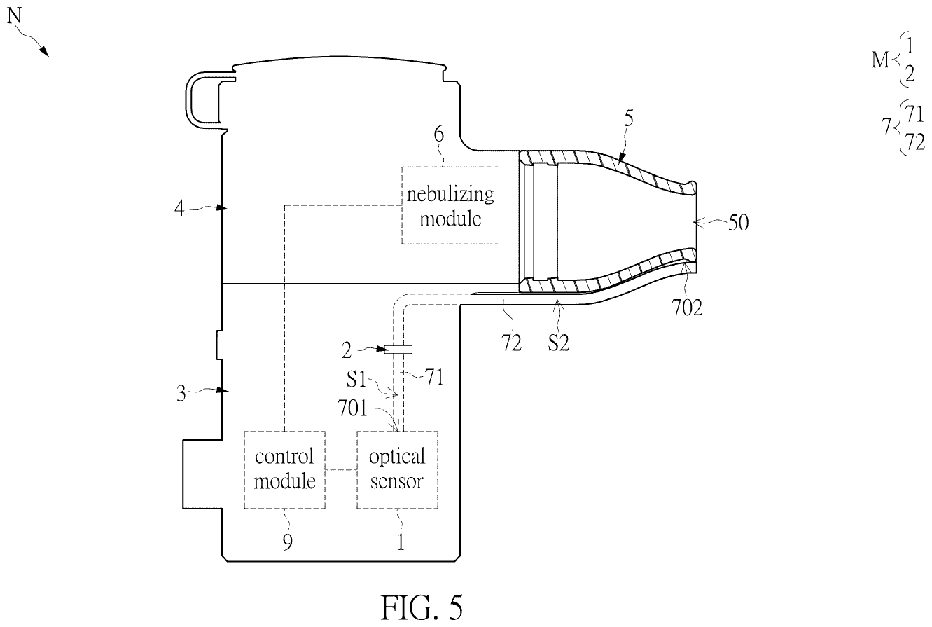

The specific implementation of the nebulizer N is not limited in the present disclosure. Reference is made to FIG. 5. The present disclosure provides another nebulizer N, in which the optical sensing module M is also applicable. Specifically, the nebulizer N shown in FIG. 5 includes the optical sensing module M that includes the optical sensor 1 and the membrane structure 2, the host 3, the cup body 4, the nozzle assembly 5, and the nebulizing module 6. Furthermore, the nebulizer N shown in FIG. 5 further includes a hollow pipe 7. The hollow pipe 7 is attached to an outer surface of the nozzle assembly 5. The hollow pipe 7 includes a connection opening 701 and a detection opening 702. The connection opening 701 is connected to the optical sensor 1, and the detection opening 702 is exposed and adjacent to the nozzle 50.

The hollow pipe 7 can be a multi-piece pipe assembly. For example, as shown in FIG. 5, the hollow pipe includes a first part 71 and a second part 72. The membrane structure 2 blocks between the first part 71 and the second part 72. The first part 71 forms a detection channel S1 between the membrane structure 2 and the optical sensor 1. The second part 72 forms a gas diffusion channel S2 between the membrane structure 2 and the detection opening 702. The gas diffusion channel S2 and the detection channel S1 are separated from and not in fluid communication with each other.

In FIG. 5, the nozzle 50 is aligned with the detection opening 702; therefore, the pressure change of the air flow near the detection opening 702 of the hollow pipe 7 is equivalent to the pressure change of the nozzle 50. When the user holds the nozzle assembly 5 in the mouth and inhales, both of the detection opening 702 and the nozzle 50 generate negative pressure. Since the pressure change of the nozzle 50 is equivalent to the pressure change of the detection opening 702, the deformation of the membrane structure 2 caused by the pressure change of the detection opening 702 is equivalent to the deformation of the membrane structure 2 caused by the pressure change of the nozzle 50. Thereby, the optical sensor 1 can detect the deformation of the membrane structure 2 through the hollow tube 7 and output a sensing signal, and further transmit the sensing signal to the control module 9. In response to the sensing signal, the control module 9 outputs a control signal to control the activation of the nebulizing module 6.

### Beneficial Effects of the Embodiments

In the nebulizer N and the optical sensing module M provided by the present disclosure, by virtue of the membrane structure 2 being disposed in the detection hole 30 of the host 3, the optical sensor 1 can be used to detect a deformation amount of the membrane structure 2, the optical sensor 1 is used to detect a deformation amount of the membrane structure 2 to control whether the nebulizing module 6 is activated. In the nebulizer N of the present disclosure, the membrane structure 2 blocks between the nozzle 50 and the optical sensor 1, preventing the moisture in the external environment from penetrating into the inside of the nebulizer N through the detection hole 30. Moreover, in the nebulizer N of the present disclosure, the optical sensor 1 replaces the existing pressure sensor for measuring pressure changes. The optical sensor 1 is used to detect the deformation of the membrane structure 2 to determine the user's breathing status and activates the nebulizing module 6 when the user inhales.

The nebulizer N provided by the present disclosure utilizes the optical sensor 1 to detect the deformation of the membrane structure 2 to generate a corresponding sensing signal. In comparison with the pressure sensor in the existing nebulizer, there is no need to consider whether the difference in initial pressure affects the breathing detection. Moreover, the membrane structure 2 is less likely to be deformed by the user holding the nebulizer N, thereby avoiding measurement errors caused by the breathing detection function operating incorrectly.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. An optical sensing module applied to a nebulizer, the nebulizer including a nozzle, and the optical sensing module comprising:
an optical sensor disposed inside the nebulizer; and
a membrane structure disposed in the nebulizer, wherein the membrane structure blocks between the nozzle and the optical sensor;
wherein, when a user inhales against the nozzle, the membrane structure is deformed by a pressure difference generated inside the nozzle, and the optical sensor is used to detect a deformation amount of the membrane structure and output a sensing signal.

2. The optical sensing module according to claim 1, wherein the membrane structure is made of an elastomer.

3. The optical sensing module according to claim 1, wherein a thickness of the membrane structure ranges between 0.01 mm and 0.005 mm.

4. A nebulizer, comprising:
a host including a detection hole;
a cup body disposed on a top of the host;
a nozzle assembly disposed on the cup body, wherein the nozzle assembly has a nozzle;
a nebulizing module disposed in the cup body; and
an optical sensing module including:
an optical sensor disposed inside the host; and
a membrane structure disposed in the detection hole, wherein the membrane structure blocks between the nozzle and the optical sensor;
wherein, when a user inhales against the nozzle, the membrane structure is deformed by a pressure difference generated inside the nozzle, and the optical sensor is used to detect a deformation of the membrane structure and output a sensing signal.

5. The nebulizer according to claim 4, wherein the cup body includes a first housing and a second housing, the second housing includes a first connection opening, a second connection opening, and a third connection opening that are in fluid communication with each other, the first housing is disposed on the first connection opening, the nebulizing module is disposed on a bottom of the first housing, the nozzle assembly is disposed on the second connection opening, and the second housing is coupled to the host through the third connection opening.

6. The nebulizer according to claim 5, wherein the membrane structure and the nozzle have a gas diffusion channel therebetween, the membrane structure and the optical sensor have a detection channel therebetween, and the gas diffusion channel and the detection channel are separated from and not in fluid communication with each other.

7. The nebulizer according to claim 4, further comprising a hollow pipe, wherein the hollow pipe is attached to a surface of the nozzle assembly, the hollow pipe includes a connection opening and a detection opening, the connection opening is connected to the optical sensor, and the detection opening is exposed and adjacent to the nozzle.

8. The nebulizer according to claim 7, wherein the nozzle is aligned with the detection opening.

9. The nebulizer according to claim 7, wherein the hollow pipe includes a first part and a second part, the membrane structure blocks between the first part and the second part, the first part forms a detection channel between the membrane structure and the optical sensor, the second part froms a gas diffusion channel between the membrane structure and the detection opening, and the gas diffusion channel and the detection channel are separated from and not in fluid communication with each other.

10. The nebulizer according to claim 6, wherein the detection channel further includes a transparent barrier layer, and the transparent barrier layer is used to block moisture from entering the detection channel.

11. The nebulizer according to claim 9, wherein the detection channel further includes a transparent barrier layer, and the transparent barrier layer is used to block moisture from entering the detection channel.

12. The nebulizer according to claim 4, further comprising a control module electrically connected to the nebulizing module and the optical sensor, wherein the control module is used to receive the sensing signal and control the activation of the nebulizing module.
